(19) United States
(12) Patent Application Publication
(10) Pub. No.: US 2024/0221885 A1
(43) Pub. Date: Jul. 4, 2024

(54) Systems and Methods for Optimization of Disease Therapy

(71) Applicant: Fresenius Medical Care Deutschland GmbH, Bad Homburg (DE)

(72) Inventors: Luca Neri, Milan (IT); Francesco Bellocchio, Siziano (IT); Carlo Barbieri, Crema (IT)

(21) Appl. No.: 18/291,338

(22) PCT Filed: Jul. 6, 2022
(86) PCT No.: PCT/EP2022/068736

§ 371 Date: Jan. 23, 2024

(30) Foreign Application Priority Data
Jul. 7, 2021 (EP) 21184384.2

(51) Int. Cl. G16H 20/10; G16H 10/20; G16H 10/60; G16H 40/63

(52) U.S. Cl. CPC G16H 20/10; G16H 10/20; G16H 10/60; G16H 40/63

(57) ABSTRACT

A therapy optimization system for supporting therapy of diseases, particularly Mineral Bone Disorder in Chronic Kidney Disease (CKD-MBD), is provided. The system includes interfacing modules for exchanging therapy-related data with several subsystems of a clinical decision-support system (CDSS), and a therapy-support module for controlling the data exchange via the interfacing module, whereby the therapy-related data is processed by the therapy-support module to generate and to additionally transfer therapy recommendations and/or data-input requests via the interfacing modules. Also provided are a computer-implemented method for optimization of disease therapy, a computer program, and a computer program product that, when executed by a computing system, cause the system to perform the foregoing operations.

### CROSS-REFERENCE TO RELATED APPLICATIONS

The present application is the national stage entry of International Application No. PCT/EP2022/068736, filed on Jul. 6, 2022, and claims priority to Application No. EP 21184384.2, filed in the European Patent Office on Jul. 7, 2021, the disclosures of which are expressly incorporated herein in their entirety.

### TECHNICAL FIELD

The present disclosure relates to, in general, the therapy of diseases, especially the therapy of Mineral Bone Disorder in Chronic Kidney Disease (CKD-MBD), and, particularly, to a therapy optimization system for optimizing treatment outcomes, and a method and computer program to control the system.

### BACKGROUND

Medical treatment of chronic kidney disease-mineral bone disorder (CKD-MBD) is a challenging clinical problem for patients with kidney disease. CKD-MBD is a systemic disorder associated with increased cardiovascular morbidity, bone fractures and mortality risk. CKD-MBD is a complication of advanced renal disease but when effectively treated, through the normalization of the biomarkers related to mineral bone metabolism, longer survival and lower rate of cardiovascular events have been reported in clinical studies. CKD-MBD is characterized by a systemic disorder of mineral and bone metabolism generally manifested as abnormal levels of serum phosphate, serum calcium, parathyroid hormone (PTH) and serum vitamin D.

There are several different clinical interventions (e.g., drugs, diet, parotidectomy) to manage and treat this disorder but as these biomarkers characterizing the condition are highly interdependent and behave differently through different physiological pathways, correcting them and maintaining the patient with normal levels at the same time poses a clinical challenge.

Furthermore, patient behavioral patterns can influence the disorder. Although there are several guidelines on the use of drugs related to CKD-MBD, the efficacy of these treatments can be hampered by the uncertainty about patient behavior.

A complex pathophysiology, deviations from medication adherence and their effects, and contributions to the outcomes by aspects related to diet and physical activity can also impact treatment efficacy.

The clinical challenges in treating CKD-MBD can be transferred to the treatment of other diseases in which patient's behavior and medication adherence influence therapy outcomes, such as hypotension, diabetes, coronary heart/artery disease, asthma, obesity, cancer, and others.

### SUMMARY

Clinical Decision Support Systems (CDSS) have been applied to many problems in medicine. The growing availability of medical data and the advances in computer-implemented data evaluation are two of the major drivers for the development and application of such digital solutions within CDSS. CDSS can be very effective in supporting clinicians and other involved parties in the treatment and management of patient disorders, especially for diseases influenced by many intercorrelated factors such as CKD-MBD.

The treatment of diseases, particularly of CKD-MBD, can be optimized by exploiting advantages of technical aspects of a CDSS tailored to the underlying medical problem.

To address the stated challenges about therapy of diseases, particularly CKD-MBD, a system (such as a CDSS) can analyze, in real-time, the specificity of a patient condition, in terms of all medically contributing aspects, and adapt the management of the therapy consequently through a specific program of clinical, behavioral, and educational intervention and interactions between clinician and patient. The disclosed system can help address the multi-factorial and complex medical nature of specific diseases, particularly CKD-MBD, and the underlying physiology. The described system uses a combination of subsystems to analyze and manage subproblems concurring with CKD-MBD. The subsystems are focused on specific aspects of the disease (e.g., medication adherence, patient dose-response relation, diet, physical activity, clinical intervention), while a central element of the system combines and processes the different subsystems' inputs and outputs to optimize a strategy of treatment.

In the following, the description is focused on the therapy of CKD-MBD. The transferability of the described techniques to other diseases in which the patient's behavior and medication adherence significantly influence the therapy outcomes (such as hypotension, diabetes, coronary heart/artery disease, asthma, obesity, cancer, and others) is preserved and the application of the disclosure to those diseases is within the scope of the present disclosure.

The present system relates to supporting the therapy of CKD-MBD by personalized and real-time adaptive care interactions, which represents a holistic approach to the CKD-MBD patient. For this purpose, a digital ecosystem of subsystems is employed to address different subproblems of the medical condition. This therapy optimizer system (referred to hereinafter as a CDSS) coordinates and combines different system parts with the aim of optimizing therapy in terms of interventions and interactions.

The CDSS is adapted to provide CKD-MBD therapy support, and includes a set of interactive subsystems.

One first subsystem of the CDSS, a patient monitor, can be employed for monitoring and analyzing the medical patient status and behavior, including: medication adherence, patient activity, diet, self-assessment through sensors and electronic patient-reported outcomes. This subsystem can interact with the patient through a user interface to collect specific information (e.g., by questionnaire), to give recommendations (e.g., drug dose adjustments), and to establish interactions with clinicians (e.g. remote communications).

One second subsystem of the CDSS, a dose-response simulator, can be employed for simulating drug-dose response of a patient with a particular medical status. A dose-response relation can be computed for each CKD-MBD related drug (e.g., vitamin D, phosphate binder) by considering the specificity of the patient (e.g., biomarkers, comorbidities, lifestyle, medications).

One third subsystem of the CDSS, a clinical monitor, can be employed for patent monitoring and for evaluating clinical intervention adjustment. This subsystem can interact with the clinician through a user interface representing the general status of the patient, drug prescriptions, medication adherence, any other information useful for assessing the CKD-MBD therapy and for interacting with the patient (e.g., requesting a visit, remote communications).

The CDSS includes a CKD-MBD therapy optimizer subsystem. This subsystem can coordinate and combine the information transmitted from other subsystems to generate recommendations for the patient and clinicians. Furthermore, the CKD-MBD therapy optimizer subsystem can manage the inputs/outputs of each subsystem and the communication between patient and clinician.

According to the present disclosure, a therapy optimizer system for supporting the therapy of diseases, particularly CKD-MBD, is provided. The therapy optimizer system includes interfacing modules configured for exchanging therapy-related data with the subsystems of a CDSS adapted for CKD-MBD therapy, which include a clinical database, a clinical monitor, a dose-response simulator and a patient monitor. The therapy optimizer system further includes a therapy-support module configured to control the exchange of therapy-related data by the interfacing modules. The therapy-support module is further configured to process the transmitted therapy-related data to generate therapy-related recommendations to be additionally transmitted by the interfacing modules.

According to the present disclosure, a computer-implemented method is provided for supporting the therapy of diseases. The computer-implemented method includes: transmitting therapy-related data to interfacing modules configured for transmitting therapy-related data with a clinical database, a clinical monitor, a dose-response simulator, and a patient monitor; analyzing the therapy-related data transmitted by the interfacing modules with respect to their sufficiency for producing therapy-related recommendations; generating, based on the sufficiency analysis, data-input requests to be transmitted by the respective interfacing modules; generating, based on the therapy-related data transmitted to the interfacing modules, therapy-related recommendations to be transmitted by the respective interfacing modules; and controlling the interfacing modules to transmit respective data-input requests and/or therapy-related recommendations.

### BRIEF DESCRIPTION OF THE DRAWINGS

FIG. 1 shows a schematic depicting the components of the CDSS adapted to the therapy of CKD-MBD.

FIG. 2 shows a schematic depicting the components of the therapy optimizer subsystem of the CDSS according to the present disclosure.

FIG. 3 shows a schematic depicting data exchanged between the therapy optimizer system and other subsystems of the CDSS according to the present disclosure.

FIG. 4 shows a flowchart depicting an example process performed by the therapy optimizer system according to the present disclosure.

### DETAILED DESCRIPTION

Specific embodiments of the present disclosure are described below.

The present disclosure provides a system, a method, and a computer program to optimize therapy of CKD-MBD by coordinating the exchange of health-related data between different interfacing subsystems of a CDSS adapted to support the therapy of CKD-MBD.

The system is configured to process exchanged health-related data to generate, depending on the data exchanged, recommendations to either directly affect the contribution of a sub-system for the purpose of improved therapy outcome when implemented, and/or request additional input from the users of the respective subsystems of the CDSS.

Furthermore, the system adaptively provides current medical status and potential medical interactions to optimize therapy of CKD-MBD for involved patients and clinicians, and therefore facilitates therapy-supporting communication.

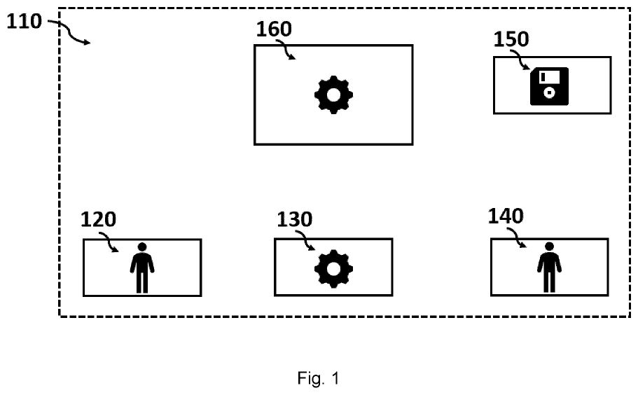

FIG. 1 shows a schematic depicting components of the CDSS adapted to the therapy of CKD-MBD.

The CDSS (110) includes a multitude of subsystems (120, 130, 140, 150).

The number of subsystems and their respective functions are adapted to the medical condition(s) for which a clinical decision is to be supported. In a general CDSS (e.g., a CDSS supporting decisions on a variety of clinical conditions), more subsystems can be included.

No limitation to spatial allocation of the subsystems is to be deduced from the depiction, since data communication between the subsystems can be established by remote data-transfer. All subsystems can also be implemented as software systems on computational hardware. In some implementations, multiple subystems or subsystem functions can be allocated to a shared pool of computational hardware resources in a distributed “cloud” environment.

The support system disclosed here is adapted to the therapy of CKD-MBD. The subsystems are adapted to reflect aspects influencing therapeutic outcomes of CKD-MBD and the monitoring of their status. For this purpose, the system can include a patient monitor (120), a clinical monitor (140), a dose-response simulator (130), a clinical data base (150), and the therapy optimizer system (160), which is provided with means for data communication to each of the other subsystems.

The patient monitor (120) can include sensors, software, and other components that collect information about the therapy-related status (e.g., symptoms, physiological measurement) and the behavior of the patient (e.g., medication adherence, diet, physical activity) from the patient. The interaction between patient and patient monitor can be conducted via a touch-sensitive display, and the system can include means for remote data exchange (e.g. via the Internet), such as a smartphone or a notebook computer, optionally connected with additional devices for physiological measurement (e.g., sensors for measurements of blood pressure and weight).

The clinical monitor (140) can include sensors, software, and other components that collectively represent a set of information about the patient, their health status and their behavior (e.g., physiological measurements, symptoms, comorbidities, diet, drugs taken, previous dose-response measurements) to a clinician or other medically versed person. The clinical monitor (140) can include a warning system that notifies the user about medical situations that require clinician attention (e.g., dangerous levels of some biomarkers) and at least one communication channel (e.g., text, voice, video) to interact with the patient synchronously or asynchronously. The interaction between clinician and clinical monitor can be conducted via touch-sensitive display, and the system can include means for remote data exchange (e.g. via the Internet), such as a smartphone or a notebook computer.

The dose-response simulator (130) can include sensors, software, and other components that collectively perform simulations about the relation between a class of drugs (e.g., vitamin D or phosphate binders for CKD-MBD) and some therapy-related biomarkers (serum calcium, phosphate, PTH). For a more accurate assessment, the simulator can include a set of information related to the patient and their behavior (physiological measurements, comorbidities, diet, drugs taken, previous dose-response measurements). The simulator can employ various models for simulating pharmacokinetics and physiological dynamics based on input of therapy-related data to simulate the dynamics of therapy-related patient biomarkers in dependence of the drugs and their respective dosing.

The clinical database (150) can include means for data storage to allow storage of electronic medical records related to patient health.

Selection of the subsystems of the CDSS (120, 130, 140, 150) from various other subsystems potentially employed in a general CDSS (and their specific combination) allow the system to technically represent the medical aspects contributing to the outcomes specific for the therapy of CKD-MBD and the condition's specific interdependence of therapeutic aspects.

The therapy optimizer system (160) can technically adapt a general CDSS to address the specific underlying processes involved in the therapy of CKD-MBD by controlling and coordinating the subsystems and by processing the data exchanged with them to improve CKD-MBD therapy outcomes.

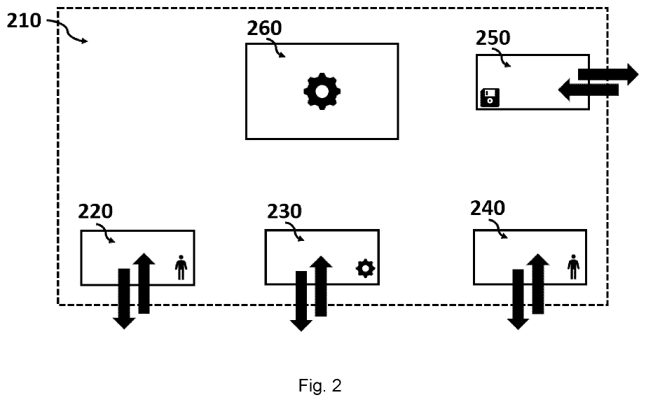

FIG. 2 shows a schematic depicting the components of the therapy optimizer subsystem of the CDSS according to the present disclosure.

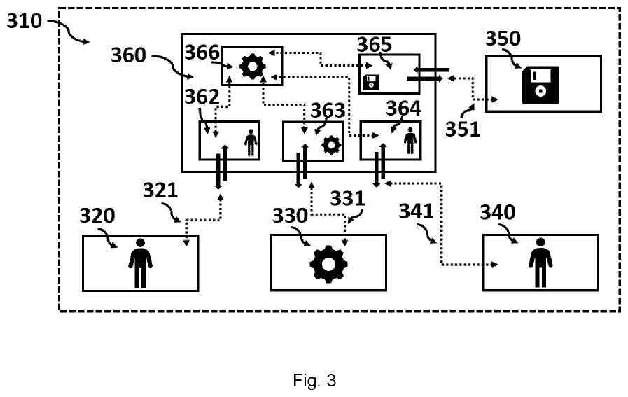

The therapy optimizer system (210), referenced by (160) in FIG. 1 and by (360) in FIG. 3, includes a multitude of modules (220, 230, 240, 250) for interfacing with subsystems of a CDSS adapted to support the therapy of CKD-MBD and a therapy-support module (260) for the coordination and control of the interfacing modules.

The selection and synergetic combination of the specific interfacing modules (220, 230, 240, 250) to be controlled by the therapy-support module (260) serves the purpose of emulating different interdependent medical aspects of the addressed condition: CKD-MBD. The interfacing module to the patient monitor (220) corresponds to patient-side contributions to treatment of the medical condition, the interfacing module to the clinical monitor corresponds to clinician-side contributions to treatment of the medical condition, and the interfacing module to the drug-dose monitor corresponds to effects of separate drugs, their dosing, and combinational effects to treatment of the medical condition. The interdependence of therapy-related contributions is addressed by the communication-coordinative therapy-support module (260).

In one aspect of the present disclosure, the therapy-support module (260) is further configured to process therapy-related data transmitted via the interfacing modules (220, 230, 240, 250). This allows for the generation of therapy-related recommendations to be additionally transmitted by the interfacing modules (220, 230, 240, 250). Different recommendations can be generated and transmitted by the respective interfacing modules (220, 230, 240, 250) corresponding to different therapy-related aspects that can be differently addressed (e.g. by clinician, patient, and drug-dosing). This allows for the optimization of the patient's therapy outcome as result of recommended changes to the respective contributions.

FIG. 3 shows a schematic depicting data exchanged between the therapy optimizer and other subsystems of the CDSS according to an embodiment of the disclosure.

The CDSS (310) is adapted to support the therapy of CKD-MBD, and includes the following subsystems: patient monitor (320), clinical monitor (340), drug-dose simulator (330), and clinical database (350), which are in respective data communication (321, 331, 341, 351) to the therapy optimizer system (360) via interfacing modules (362, 363, 364, 365) included in the therapy optimizer system (360), which communicates with the therapy-support module (366).

In one first aspect of the present disclosure, the interfacing module (365) to the clinical database is configured to exchange data (351) related to patient health records with a clinical database (350). This allows the system to import therapy-related data from medical records stored in the clinical database (350) via the interfacing module (365) for consideration of CKD-MBD therapy outcomes by the therapy-support module (366). Recommended therapy adaptions and effects of actions performed by patient and/or clinicians resulting from treatment recommendations can be recorded to the clinical database (350). In this way, medical records can be updated and iteratively imported to the therapy optimizer system (360) to allow for an adaptive and continuous “on-line” therapy optimization.

In one second additional or alternative aspect of the present disclosure, the interfacing module (364) to the clinical monitor is configured to exchange data (341) related to one or more of drug-dose adjustments, visit assessments, medical patient needs and one or more of clinical patient characteristics, biomarkers, drug treatments, medical warnings, or support requests with a clinician-used clinical monitor (340) via the interfacing module (364) for consideration of CKD-MBD therapy outcomes by the therapy-support module (366). Therapy adaptions provided by the therapy optimizer system (360) that involve assessment and/or decision by a clinician can be communicated to the user of the clinical monitor (340). A clinician's recommendation for therapy and/or reactions to the patient's medical status and development can be fed back to the therapy optimizer system (360) to allow an adaptive clinician-side contribution to therapy support.

In one third additional or alternative aspect of the present disclosure, the interfacing module (362) to the patient monitor is configured to exchange data (321) related to one or more of drug-dose adjustments, physical activity, visit assessments, or a questionnaire for clinical data and one or more of drug intake, diet, physical activity, patient-reported outcomes, or sensor measurements with a patient-used patient monitor (320) via the interfacing module (362) for consideration of CKD-MBD therapy outcomes by the therapy-support module (366). Therapy adaptions provided by the therapy optimizer system (360) involving execution by the patient can be communicated to the user of the patient monitor (320). A patient's reporting on their physiological parameters, drug adherence, and/or physical activity can be fed back to the therapy optimizer system (360) to allow an adaptive patient-side contribution to the therapy support.

In one fourth additional or alternative aspect of the present disclosure, the interfacing module (363) to the drug-dosing simulator is configured to exchange data (331) related to a patient's dose-response relation and one or more of drug intake, diet, biomarkers, or patient characteristics with a drug dose-response simulator (330) via the interfacing module (363) for consideration of CKD-MBD therapy outcomes by the therapy-support module (366). Physiological and drug-related aspects of the therapy provided by the therapy optimizer (360) used for the simulation of the patient's drug response can thus be communicated to the drug-response simulator (330). The results of the simulation in terms of drug dosing as well as the quantity and quality for best therapy outcomes (e.g., improvement of patient-characteristic biomarkers), in addition to variance imposed by non-drug aspects of the therapy (e.g. diet, physical activity) can be fed back to the therapy optimizer system (360) to allow an adaptive drug-response contribution to therapy support.

The foregoing aspects of the present disclosure can be employed in any combination to satisfy the constraints of the underlying CDSS and to emulate contributions to the specific CKD-MBD therapy. The additional combination with further interfacing modules to other CDSS subsystems can provide further potential for therapy improvement. Particular advantages can be found in the combination of these aspects, without further configuration of the interfacing modules to a clinical monitor or a clinical database, for therapy cases, in which therapy outcomes are mainly dependent on patient behavior and drug dosing (adherence), with or without clinical contributions. Additional or alternative particular advantages can be realized without further configuration of the interfacing modules to a drug-dosing simulator or a clinical database, for therapy cases, in which the interactions between clinician and patient (e.g. by regular checkups) contribute to the therapy outcomes, with or without drug dosing (adherence). Furthermore, additional or alternative particular advantages can be realized without specific configuration of the interfacing modules to a patient monitor for cases which are not specific for a particular patient, but are mostly influenced by drug-dosing with respect to medical records (e.g. in the frame of statistical drug-response data analytics).

The interfacing modules (362, 363, 364, 365) of the therapy optimizer system (360) can be configured to exchange treatment-related data (321, 331, 341, 451) with subsystems of the CDSS. The subsystems may each be configured to perform individual tasks independent of their combined employment in a CDSS adapted to optimize the therapy of CKD-MBD, so a data formatting and processing specifically suited to that respective task may be different for each subsystem. For instance, the clinical database (350) may format the data within a clinic-specific health-record form sheet or a data format suited for improved exchange between different clinics via a certain compressing or indexing system. For instance, the drug-dosing simulator (330) may format the data, in terms of model variables and their ranges, for improved importing into the algorithmic evaluation underlying the simulation. Since the therapy module (366) of the therapy optimizer system (360) combines the therapy-related data and another data format may be preferable for this type of data processing, it may therefore be desirable to transform the data exchanged via the interfacing modules (362, 363, 364, 365). Therefore, in one aspect of the present disclosure, the interfacing modules (362, 363, 364, 365) are further configured to transform therapy-related data exchanged by the interfacing modules between a data format used in the processing of the therapy-support module (366) and a data format of the data transmitted to the respective interfacing modules. The transformation can include re-allocation of the data-structure parts, their combination, conditional filtering, and other methods suitable to adapt the format in which the data is exchanged and processed.

The processing of the therapy-related data by the therapy-support module (366) of the therapy optimizer system (360) can include aggregating the data by controlling the interfacing modules (362, 363, 364, 365) to transfer data from the CDSS subsystems. The aggregated data can then be pre-analyzed for data relevant for the therapy of CKD-MBD as preconfigured in definitions. At this pre-processing step, redundant information contained in the data transferred from different subsystems, as well as information irrelevant to the therapy of CKD-MBD, can be filtered out before being processed by the therapy-support module (366). This reduces the processing overhead of the module.

According to one aspect of the present disclosure, the treatment-related data can be analyzed within the processing of the therapy-support module (366) with respect to their sufficiency for generating therapy-related recommendations. The sufficiency analysis can be performed by matching the aggregated data to preset definitions of data required for sufficiency. Here, sufficiency for generating therapy-related recommendations can be defined by different aspects of the therapy (e.g., biomarkers, drug dosing, diet, physical activity) and their respective representation by the aggregated data. In one embodiment, sufficiency can be established by a complete set of information related to the patient's medical history in terms of drug intake if the only contribution to the patient's specific CKD-MBD therapy is on the drug-dosing side. In another embodiment, sufficiency can be established by a complete set of information related to the patient's physical activity and medication adherence, the clinician's prescription, and the minimum requirement of the drug-response simulator to produce a recommended drug dosing. The sufficiency criterion is not necessarily established only if the amount of available therapy-related data exceed a certain degree of potentially available or required data (e.g. 60% of input data required by the drug-response simulation), but can also be established if the minimum required data could be derived from the available therapy-related data by derivation or deduction. Means of data derivation/deduction can include algorithmic analysis performed by artificial neural networks, and may utilize deep learning, Bayesian networks, evolutionary algorithms, and others. The sufficiency analysis can advantageously prevent recommendations based on insufficient data, and therefore directly contributes to the quality of the resulting therapy optimization, as well as to the efficiency of the process.

In one aspect of the present disclosure, the therapy-support module (366) is configured to, based on the sufficiency analysis, control the interfacing modules (362, 363, 364, 365) to request additional data-input from the CDSS subsystems, for example, by generating and transmitting data-input requests via the interfacing modules (362, 363, 364, 365). This operation can be performed if the therapy-related data aggregated is insufficient for generating therapy-related recommendations. The control of the therapy-support module can include identifying, from matching the aggregated data to preset definitions of data sufficiency constraints, subsets of data missing for sufficiency. The process can further include identifying which interfacing module(s) are suitable to request the subsets of data missing for sufficiency. The requests for additional data-input can contain indexes of the missing data subsets. The therapy-support module generates data-input requests considering the sufficiency analysis, missing data subsets, and suitable interfacing modules. The requests can be transformed by the reformatting process of the interfacing modules to provide the request in a data format used by the subsystem with which the interfacing module is exchanging data. The therapy-support module's control of the interfacing modules to transmit the generated data-input requests allows for sequential improvement of the CKD-MBD therapy, particularly for cases in which initially provided therapy-related data is insufficient for generating recommendations. Including a common timestamp criterion of aggregated therapy-related data for the consideration for sufficiency allows the data-input requests to be generated if certain data sub-sets are outdated, which allows the therapy optimization to be performed in a continuous and adaptive manner. This is desirable even if the aggregated data set is complete with respect to sufficiency for a recommendation.

According to one aspect of the disclosure, if the data aggregated by the therapy-support module (366) is sufficient, the therapy-support module (366) is further configured to generate, based on the therapy-related data (321, 331, 341, 451) transmitted to the interfacing modules (362, 363, 364, 365), therapy-related recommendations to be transmitted by the respective interfacing modules (362, 363, 364, 365). The contents of therapy-related recommendations can be identified by aspects of the patient's specific CKD-MBD therapy in terms of the status of the therapy parameters and potential for improvement as assessed by the clinician and as indicated by the drug-response simulation. The influence of the patient's contributions on the therapy (e.g., medication adherence, diet, physical activity) specific for the medical condition CKD-MBD can make the patient a target of such recommendations. In one embodiment, depending on the patient's change in diet, the therapy-support module (366) might identify a difference between the patient's current drug dosing and the one resulting as optimal from a drug-response simulator (330) according to the change of the patient's diet and other physiological parameters obtained from a clinical database (350). In such cases, the identified difference in drug-dosing would be employed by the therapy support-module (366) to generate a recommendation to the patient to be transferred via the interfacing module (362) to the patient monitor (320), preferably advising the user of the patient monitor (320) to adapt the drug dose in accordance with the format used by the patient monitor. In another embodiment, if the aggregated therapy parameters exceed values defined to be critical, a medical warning may be triggered for the specific patient by the clinician interfacing with a clinical monitor (340). In this case, the therapy-support module (366) can generate a recommendation with the content of the critical parameters and control the interfacing module (364) to include the recommendation into the therapy-related data (341) exchanged with the clinical monitor (340), preferably advising the user of the clinical monitor (340) to check the critical value in the format used by the clinical monitor and/or request the user to appoint a checkup with the patient. Additionally, the therapy-support module (366) can control the interfacing module (365) to the clinical database (350) to transfer generated treatment-related recommendations to the clinical database (350) for storage and integration into the patient's health records. The generation of therapy-related recommendations by the therapy-support module (366) and their transfer to the respective CDSS subsystems by the interfacing modules (362, 363, 364, 365) allows the system to prompt changes for optimizing the CKD-MBD therapy specific to the patient that are relying on the system's user to be executed. The identification of recommendation content and their distribution to the interfacing modules interfacing modules (362, 363, 364, 365) by the therapy-support module (366) helps address the interdependence of CKD-MBD therapy aspects by specifically accounting for therapy aspects contributing to the improvement of clinical outcomes via separate modules. In this way, direct contributors to therapy can be separated and addressed sequentially in a manner adaptive to the dynamics of the patient's medical status.

In one aspect of the disclosure, the generated requests for additional data-input can be part of the therapy-related recommendations generated by the therapy-support module (366). This may be preferable in situations where the aggregated data are insufficient to generate recommendations with respect to some aspects of the CKD-MBD therapy, but sufficient to generate recommendations with respect to other aspects. This can also be preferable in situations where the aggregated data are sufficient to generate recommendations, but the statistical uncertainty of the underlying data and/or the resulting recommendations exceeds a preset value. A measure for the matching of the aggregated data to the constraints for a statistically precise recommendation with regards to all contributing therapy aspects is the recommendation accuracy. The recommendation accuracy can be additionally incorporated into the sufficiency criterion (e.g., the accuracy has to exceed a required pre-set value) for generating requests for additional data-input and/or therapy-related recommendations. The recommendation accuracy can also be included as part of the generated recommendation. Inclusion of the recommendation accuracy for sufficiency and/or as part of the generated recommendations allows for an additional quality measure of the therapy optimization. Additionally, reducing the likelihood of generating inaccurate recommendations by the therapy-support module (366) and their transfer via the interfacing modules (362, 363, 364, 365) can reduce the processing and data transfer load on the system.

According to one aspect of the disclosure, the minimum recommendation accuracy is set according to additional accuracy-setting (e.g. user-input) data transferred to the interfacing modules (362, 363, 364, 365). This allows the pre-set recommendation accuracy to be adapted with respect to the dynamics of the optimized CKD-MBD therapy. This additionally allows the users of the CDSS's subsystems to include feedback corresponding to the quality of the recommendations generated by the therapy-support module (366) into the data (321, 341, 351) exchanged via the interfacing modules. This contributes to adaptive algorithmic evaluations (e.g., reinforcement learning) performed as part of the system's processing.

Since the therapy optimizer system (360) can be implemented by software means on a computational system, one aspect of the disclosure defines the computer-implemented method to control the processes performed by the therapy optimizer system (360). The method includes: transmitting therapy-related data to the interfacing modules; analyzing the therapy-related data transmitted by the interfacing modules with respect to their sufficiency for producing therapy-related recommendations; generating, based on a sufficiency analysis, data-input requests to be transmitted by the respective interfacing modules; generating, based on the therapy-related data transmitted to the interfacing modules, therapy-related recommendations to be transmitted by the respective interfacing modules; and controlling the interface modules to transmit respective data-input requests and/or therapy-related recommendations.

The therapy optimizer system's configuration to perform therapy optimization in an adaptive and continuous manner, as outlined in various aspects of the present disclosure, translates to continuity and adaptiveness of the computer-implemented method. The foregoing operations of the method can be interchanged in order and/or iterated or omitted in some embodiments.

In one embodiment of the disclosure, a computer program includes instructions that, when executed by a computer, cause the computer to carry out the method performed by the therapy optimizer system.

In one embodiment of the disclosure, a computer program product includes instructions for performing the method performed by the therapy optimizer system.

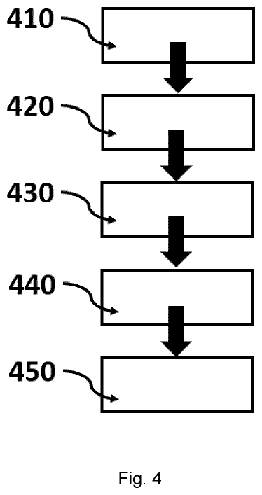

FIG. 4 shows a flowchart depicting an example process performed by the therapy optimizer system according to the present disclosure.

One example process is initiated by the therapy optimizer system's interfacing module to the patient monitor receiving (410) data related to the increase of phosphate consumption by the patient's dietary changes. The therapy-support module receives the data and determines that the data is insufficient for generating therapy-related recommendations. Therefore, the therapy support module generates requests for additional data inputs to be transferred (420) by the interfacing modules, for example, to request therapy-related data concerning the patient's actual drug dose from the clinical data base and to request therapy-related data concerning biomarkers from the clinical monitor. Therapy-related data received via the interfacing modules as result of the requests are combined with the initial data related to patient diet by the therapy-support module, and the aggregated data is transferred (430) to the interfacing module to the drug-response simulator of the CDSS with a request generated by the therapy-support module for a set of values of CKD-MBD biomarkers, considering variation on phosphate binders and related drugs. The drug-response simulator results are transmitted via the interfacing module to the therapy-support module, where based on the transmitted data, an adaption of the patient's drug dosing is employed to generate a recommendation for therapy optimization (440). The therapy-support module controls the interfacing modules to transmit (450) the therapy-related recommendation to the patient monitor (in the form of advice to change drug dosing) to the clinical monitor (in the form of a notification concerning changes in the patient's CKD-MBD therapy), and to the clinical database (in the form of an update to the patient's medical records).

1-15. (canceled)

16. A therapy optimizer system, the therapy optimizer system comprising:
interfacing modules configured for transmitting therapy-related data with a clinical data base, a clinical monitor, a dose-response simulator, and a patient monitor; and
a therapy-support module configured to control the transmission of therapy-related data by the interfacing modules,
wherein the therapy-support module is further configured to process the transmitted therapy-related data to generate therapy-related recommendations to be additionally transmitted by the interfacing modules.

17. The therapy optimizer system according to claim 16, wherein the system is configured to support a therapy of a disease comprising CKD-MBD, hypotension, diabetes, coronary heart/artery disease, asthma, obesity, or cancer.

18. The therapy optimizer system according to claim 16, wherein one of the interfacing modules is further configured to transmit data related to patient health records with a clinical database.

19. The therapy optimizer system according to claim 16, wherein one of the interfacing modules is further configured to transmit data related to one or more of drug-dose adjustments, visit assessments, or medical patient constraints and one or more of clinical patient characteristics, biomarkers, drug treatments, medical warnings, or support requests with a clinician-used clinical monitor.

20. The therapy optimizer system according to claim 16, wherein one of the interfacing modules is further configured to transmit data related to a patient's dose-response relation and one or more of drug intake, diet, biomarkers, or patient characteristics with a drug dose-response simulator.

21. The therapy optimizer system according to claim 16, wherein one of the interfacing modules is further configured to transmit data related to one or more of drug-dose adjustments, physical activity, visit assessments, or a questionnaire for clinical data and one or more of drug intake, diet, physical activity, or patient-reported outcomes and sensor measurements with a patient-used patient monitor.

22. The therapy optimizer system according to claim 16, wherein the interfacing modules are further configured to transform therapy-related data transmitted by the interfacing modules from a data format used in the processing of the therapy-support module to a data format of the data transmitted to the respective interfacing modules.

23. The therapy optimizer system according to claim 16, wherein the therapy-support module is further configured to analyze the therapy-related data transmitted to the interfacing modules with respect to their sufficiency for generating therapy-related recommendations.

24. The therapy optimizer system according to claim 23, wherein the therapy-support module is further configured to generate data-input requests to be transmitted by the respective interfacing modules when the therapy-related data transmitted to the interfacing module are insufficient for generating therapy-related recommendations.

25. The therapy optimizer system according to claim 23, wherein the therapy-support module is further configured to generate, based on the therapy-related data transmitted to the interfacing modules, therapy-related recommendations to be transmitted by the respective interfacing modules when the therapy-related data transmitted by the interfacing modules are sufficient for generating therapy-related recommendations.

26. The therapy optimizer system according to claim 25, wherein sufficiency criterion for the analysis of the therapy-related data transmitted to the interfacing modules is related to a pre-set recommendation accuracy and the generated therapy-related recommendations additionally include the recommendation accuracy and the generated data-input requests to be transmitted by the respective interfacing modules.

27. The therapy optimizer system according to claim 26, wherein the recommendation accuracy is set according to additional data transmitted to the interfacing module.

28. A computer-implemented method for supporting the therapy of diseases, the method comprising:
transmitting therapy-related data to interfacing modules configured for transmitting therapy-related data to a clinical database, a clinical monitor, a dose-response simulator, and a patient monitor;
analyzing the therapy-related data transmitted by the interfacing modules with respect to their sufficiency for producing therapy-related recommendations;
generating, based on the sufficiency analysis, data-input requests to be transmitted by the respective interfacing modules;
generating, based on the therapy-related data transmitted to the interfacing modules, therapy-related recommendations to be transmitted by the respective interfacing modules; and
controlling the interfacing modules to transmit respective data-input requests and/or therapy-related recommendations.

29. The computer-implemented method according to claim 28, wherein one of the interfacing modules is further configured to transmit data related to patient health records with the clinical database.

30. The computer-implemented method according to claim 28, wherein one of the interfacing modules is further configured to transmit data related to one or more of drug-dose adjustments, visit assessments, or medical patient constraints and one or more of clinical patient characteristics, biomarkers, drug treatments, medical warnings, or support requests with the clinical monitor.

31. The computer-implemented method according to claim 28, wherein one of the interfacing modules is further configured to transmit data related to a patient dose-response relation and one or more of drug intake, diet, biomarkers, or patient characteristics with the dose-response simulator.

32. The computer-implemented method according to claim 28, wherein one of the interfacing modules is further configured to transmit data related to one or more of drug-dose adjustments, physical activity, visit assessments, or a questionnaire for clinical data and one or more of drug intake, diet, physical activity, or patient-reported outcomes and sensor measurements with the patient monitor.

33. The computer-implemented method according to claim 28, wherein the interfacing modules are further configured to transform the therapy-related data transmitted by the interfacing modules from a data format used in the processing of a therapy-support module to a data format of the data transmitted by the respective interfacing modules.

34. The computer-implemented method according to claim 28, wherein the therapy-support module is further configured to analyze the therapy-related data transmitted by the interfacing modules with respect to their sufficiency for generating therapy-related recommendations.

35. A computer program comprising instructions which, when executed by a computer, cause the computer to perform operations comprising:
transmitting therapy-related data to interfacing modules configured for transmitting therapy-related data to a clinical database, a clinical monitor, a dose-response simulator, and a patient monitor;
analyzing the therapy-related data transmitted by the interfacing modules with respect to their sufficiency for producing therapy-related recommendations;
generating, based on the sufficiency analysis, data-input requests to be transmitted by the respective interfacing modules;
generating, based on the therapy-related data transmitted to the interfacing modules, therapy-related recommendations to be transmitted by the respective interfacing modules; and
controlling the interfacing modules to transmit respective data-input requests and/or therapy-related recommendations.
